(19) United States
(12) Patent Application Publication
(10) Pub. No.: US 2011/0196397 A1
(43) Pub. Date: Aug. 11, 2011

(54) MEDICAL DEVICE WITH A GUIDEWIRE FOR PENETRATING OCCLUSIONS

(75) Inventors: Mark G. Frantz, New York, NY (US); Stephanie A.S. Harrington, Mentor, OH (US); Ran Carmeli, Rinatya (IL); Itai Yonat, Tel Aviv (IL); Paul L. Erickson, Eastlake, OH (US)

(73) Assignee: VASOSTAR, INC., Mentor, OH (US)

(21) Appl. No.: 13/123,637

(22) PCT Filed: Oct. 9, 2009
(86) PCT No.: PCT/US09/60081

§ 371 Date: Apr. 11, 2011

Related U.S. Application Data
(60) Provisional application No. 61/104,584, filed on Oct. 10, 2008.

(51) Int. Cl. A61B 17/22

(52) U.S. Cl. 606/159

(57) ABSTRACT

A medical device includes an outer catheter having a longitudinal axis, a movable device that can pass through the catheter and is movable along the longitudinal axis relative to the catheter, and a magnetic drive engine toward a distal end of the catheter. The magnetic drive engine includes at least two components with respective magnetic field-generating coils and magnets, that move relative to one another. One of the two components is coupled to the movable device for driving vibratory motion in the movable device along the longitudinal axis. An exemplary movable device is a guidewire over which the catheter is fed. The device typically includes a second catheter threaded inside the first catheter. The first or outer catheter shields the magnetic engine and its moving components.

This application claims the benefit of U.S. Provisional Patent Application No. 61/104,584, filed Oct. 10, 2008, which is incorporated herein by reference.

### FIELD OF THE INVENTION

The present invention is related to a medical device, in particular a medical device that uses a guidewire to guide other devices to remote locations in the body of a patient, and more particularly to a medical device that is used to navigate vascular systems and/or to open blockages in a vascular system.

### BACKGROUND

In some medical procedures a guidewire is used to guide a larger device to a remote location in a vascular system of a patient. Guidewires generally are small enough and flexible enough that a surgeon can maneuver the guidewire through a blood or lymphatic vessel, without damaging the vessel walls. Typically, the guidewire is inserted into a patient's vascular system via an incision and advanced through a vessel to the desired location. The guidewire thus defines a path to that location. The surgeon can then advance a catheter or other device (such as a balloon catheter or stent, for example) over the guidewire, using the guidewire as a rail to reach the desired location in the vessel.

Sometimes the vessel is partially or completely blocked and a passage must be opened through the blockage, medically referred to as an occlusion, to reach the other side of the blockage. In angioplasty, for example, a guidewire is used to guide a catheter over the guidewire to the blockage, and a balloon at the end of the catheter is expanded to open the passage and substantially open the vessel. Before the balloon can be expanded, however, a passage has to be opened in the blockage so that the balloon can be inserted.

Sometimes the blockage is made of a soft material or only partially blocks the passage through the vessel and the surgeon can easily push the guidewire through the blockage. When the vessel is completely blocked by a harder material, the surgeon has more difficulty pushing the flexible guidewire through the blockage without damaging the walls of the vessel.

### SUMMARY

The present invention provides a medical device that uses a magnetic engine to drive oscillatory motion in an element, such as a guidewire, to assist a surgeon in opening a passage through a blockage in a vessel of a vascular system. This device also can facilitate maneuvering a guidewire to the desired location in the patient's body. In an exemplary embodiment, the guidewire passes through an inner catheter that is itself telescoped within an outer catheter. We have found that by using a catheter-within-a-catheter we can separate the magnetic engine from the vessel walls. This helps to prevent or minimize damage to those walls that might be caused by operation of the engine.

The present invention also provides a magnetic device that works with a guidewire of a surgeon's choosing. It generally is helpful for the surgeon to be able to feel the resistance created by the blockage and the vessel walls through the guidewire so that the vessel walls are not damaged while attempting to push through the blockage. Surgeons generally prefer the feedback provided by a particular type of guidewire. We have found that by selectively coupling the inner catheter to the guidewire, almost any guidewire can be used, thereby preserving the sensory feedback that a surgeon prefers from a particular type of guidewire.

Another advantage provided by the invention lies in an embodiment of a medical device with a stop in the engine. We have found it advantageous to use a stop to maintain an active zone by limiting the distance that the components of a magnetic drive engine can move relative to one another. The active zone is defined by overlapping longitudinal portions of the components of the magnetic drive engine, specifically magnets and magnetic field-generating coils, and more specifically by the overlapping magnetic fields that they provide.

More particularly, an exemplary apparatus provided by the invention includes an outer catheter having a longitudinal axis, a movable device that can pass through the catheter and is movable along the longitudinal axis relative to the catheter, and a magnetic drive engine toward a distal end of the catheter that includes at least two components that move relative to one another. One of the two components is coupled to the movable device for driving vibratory motion in the movable device along the longitudinal axis.

According to another feature provided by the invention, the components of the engine have longitudinally-overlapping portions that define an active zone and a stop is positioned to maintain the active zone when the movable device is displaced in a distal direction.

Another medical device provided by the invention includes an outer catheter having a longitudinal axis, an inner catheter telescopically inserted in the outer catheter, a guidewire telescopically inserted in the inner catheter, and a magnetic drive engine toward a distal end of the outer catheter to vibrate the guidewire along the longitudinal axis.

The present invention also provides a medical device including an outer catheter having a longitudinal axis, an inner catheter telescopically inserted in the outer catheter, a guidewire telescopically inserted in the inner catheter, and a magnetic drive engine toward a distal end of the outer catheter to vibrate the guidewire along the longitudinal axis. In this case, the inner catheter is coupled to the guidewire for longitudinal movement with the guidewire relative to the outer catheter.

The foregoing and other features of the invention are hereinafter fully described and particularly pointed out in the claims, the following description and annexed drawings setting forth in detail certain illustrative embodiments of the invention, these embodiments being indicative, however, of but a few of the various ways in which the principles of the invention may be employed.

### BRIEF DESCRIPTION OF THE DRAWINGS

FIG. 1 is a schematic longitudinal cross-section of a guidewire.

FIG. 2 is a schematic longitudinal cross-section of a catheter

FIG. 3 is a schematic longitudinal cross-section of a medical device composed of a guidewire with magnets telescopically inserted in a catheter with magnetic field-generating coils.

FIG. 4A is a schematic longitudinal cross-section of alternative medical device with magnetic field-generating coils on a guidewire and magnets on a catheter.

FIG. 4B is a schematic longitudinal cross-section of the guidewire of FIG. 4A.

FIG. 4C-1 is a schematic longitudinal cross-section of the catheter of FIG. 4A.

FIG. 4C-2 is a schematic longitudinal cross-section of a magnet of FIG. 4A.

FIG. 5A-1 is a schematic longitudinal cross-section of a catheter with magnetic field-generating coils and a treatment device carried thereon.

FIG. 5A-2 is a schematic side view of a guidewire with magnetic beads coupled to the guidewire.

FIG. 5A-3 is a schematic longitudinal cross-section of another catheter.

FIG. 5B is a schematic longitudinal cross-section of a medical device that includes the combination of the guidewire of FIG. 5A-2 inside the catheter of FIG. 5A-1 within catheter of FIG. 5A-3.

FIG. 5C is another schematic section of the medical device of FIG. 5B.

FIG. 6A-1 is a partial schematic longitudinal cross-section of another catheter and treatment device.

FIG. 6A-2 is a schematic longitudinal cross-section of a guidewire.

FIG. 6A-3 is a schematic longitudinal cross-section of a catheter.

FIG. 6B is a schematic longitudinal cross-section of the guidewire of FIG. 6A-2 within the catheter of FIG. 6A-1 which is inside the catheter of FIG. 6A-3.

FIG. 6C is another schematic section of the medical device of FIG. 6B.

FIG. 7A-1 is a schematic partial longitudinal cross-section of a catheter with a treatment device supported thereby.

FIG. 7A-2 is a schematic side view of a guidewire.

FIG. 7A-3 is a schematic longitudinal cross-section of a catheter with magnetic field-generating coils therein.

FIG. 7B is a schematic longitudinal cross-section of a medical device that includes the combination of the guidewire of FIG. 7A-2 inside the catheter of FIG. 7A-1 within the catheter of FIG. 7A-3.

FIG. 7C is a partial schematic section of the medical device of FIG. 7B.

FIG. 8A-1 is a schematic longitudinal cross-section of a catheter.

FIG. 8A-2 is a schematic partial longitudinal cross-section of a guidewire.

FIG. 8A-3 is a schematic longitudinal cross-section of a catheter with ring magnets.

FIG. 8B is a schematic longitudinal cross-section of a medical device that includes the combination of the guidewire of FIG. 8A-2 inside the catheter of FIG. 8A-1 which is inside the catheter of FIG. 8A-3.

FIG. 8C is a schematic section of the medical device of FIG. 8B.

FIG. 9A-1 is a schematic longitudinal cross-section of a catheter.

FIG. 9A-2 is a schematic partial longitudinal cross-section of a guidewire.

FIG. 9A-3 is a schematic longitudinal cross-section of a catheter with ring magnet pairs.

FIG. 9B is a schematic longitudinal cross-section of a medical device that includes the combination of the guidewire of FIG. 9A-2 inside the catheter of FIG. 9A-1 which is inside the catheter of FIG. 9A-3.

FIG. 9C is a schematic section of an alternative catheter for use in the medical device of FIG. 9B.

FIG. 10A-1 is a schematic longitudinal cross-section of a guidewire.

FIG. 10A-2 is a schematic longitudinal cross-section of a catheter carrying a treatment device.

FIG. 10A-3 is a schematic longitudinal cross-section of another catheter.

FIG. 10B is a schematic longitudinal cross-section of a medical device that includes the combination of the guidewire of FIG. 10A-1 inside the catheter of FIG. 10A-2 which is inside the catheter of FIG. 10A-3.

FIGS. 11A-11E are schematic longitudinal sections of exemplary catheters for use in medical devices provided by the present invention.

FIG. 12A is a schematic longitudinal cross-section of a medical device having a guidewire inside an inner catheter which is inside an outer catheter.

FIG. 12A-1 is an enlarged section of FIG. 12A.

FIGS. 12B is a schematic longitudinal cross-section of a medical device having a guidewire inside an inner catheter which is inside an outer catheter.

FIG. 12B-1 is an enlarged section of FIG. 12B.

FIG. 13A is a schematic longitudinal section of a piston portion of another medical device provided by the present invention.

FIG. 13B is a schematic longitudinal section of a catheter for use with the piston portion of FIG. 13B.

FIG. 13C is a schematic longitudinal section of an assembly of the piston portion of FIG. 13A and the catheter of FIG. 13B.

FIG. 13D is a schematic longitudinal section of a guidewire for use with the assembly of FIG. 13C.

FIG. 13E is a schematic longitudinal section of a medical device composed of the assembly of FIG. 13C and the guidewire of FIG. 13D.

FIG. 14 is a schematic longitudinal section of another medical device provided by the present invention.

### DETAILED DESCRIPTION

The present invention provides a medical device that uses a magnetic engine to drive oscillatory motion in an element, such as a guidewire, to assist a surgeon in opening a passage through a blockage in a vessel of a vascular system. A catheter or other device can then be fed over the guidewire and advanced to the desired location. In an exemplary embodiment, once the device is assembled the guidewire passes through an inner catheter that is itself telescoped within an outer catheter. We have found that by using a catheter-within-a-catheter we can separate the magnetic engine from the vessel walls. This helps to prevent or minimize damage to those walls that might be caused by operation of the engine.

The present invention also provides a magnetic device that works with a guidewire of a surgeon's choosing. Surgeons typically select a guidewire for its relative stiffness and other physical properties. It generally is helpful for the surgeon to be able to feel the resistance created by the blockage and the vessel walls through the guidewire so that the vessel walls are not damaged while attempting to push through the blockage. Surgeons generally prefer the feedback provided by a particular type of guidewire. We have found that by selectively coupling the inner catheter to the guidewire, almost any guidewire can be used, thereby preserving the sensory feedback that a surgeon prefers from a particular type of guidewire.

Another advantage provided by the invention lies in an embodiment of a medical device with a stop in the engine. We have found it advantageous to use a stop to maintain an active zone by limiting the distance that the components of a magnetic drive engine can move relative to one another. The active zone is defined by overlapping longitudinal portions of the components of the magnetic drive engine, specifically magnets and magnetic field-generating coils, and more specifically the overlapping interactive magnetic fields that the components generate.

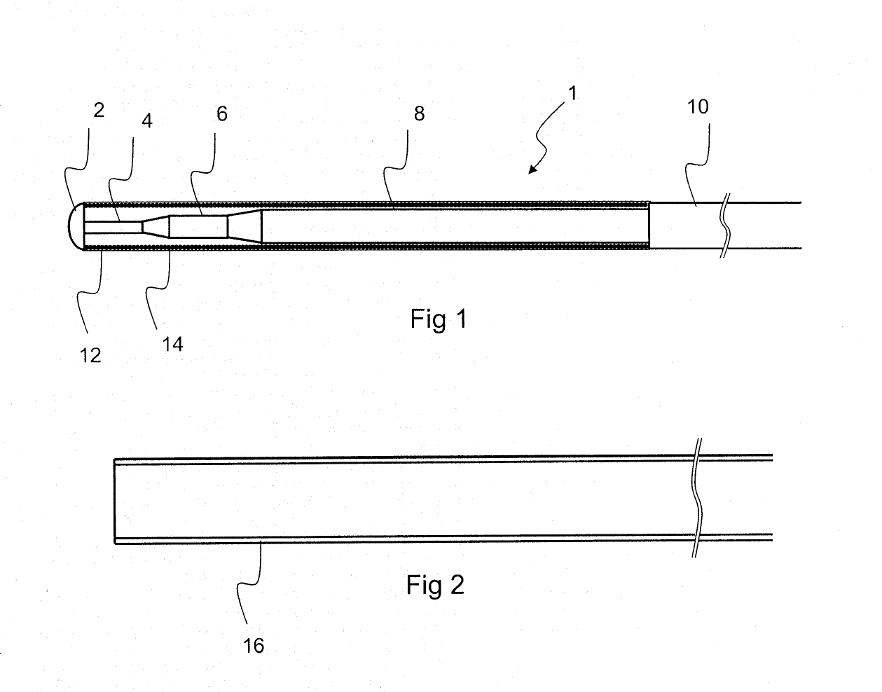

Turning now to the drawings in detail, FIG. 1 shows a typical guidewire. The guidewire 1 can be divided into a working zone 8 that is inserted into a vessel in a human body during a procedure and a body 10 that defines the remaining length of the guidewire. Once the guidewire reaches a blockage, a catheter 16 (FIG. 2) is threaded over the guidewire. The overall length of a typical guidewire ranges from about 160 centimeters to about 300 centimeters.

The working zone 8 at the leading or distal end of the guidewire 1 is divided into several segments. The distal tip 2 of the guidewire is the first segment of the guidewire 1 that engages the body of the patient and must be designed in a way that it does not unintentionally cause harm. A first core segment 4 follows the distal tip of the guidewire. The section that contains the first core segment 4 typically defines a flexible zone of the guidewire that makes it easier to maneuver through a vascular vessel. Guidewires typically have been characterized as either stiff or soft based on the nature of the guidewire in the first core segment 4. A thicker second core segment 6 follows the first core segment 4 and forms a less flexible zone also called a “stent zone.” The second core segment 6 is connected to a third core segment 8, which is thicker and stronger to help push the guidewire through the vessel. Some guidewires can include different numbers and types of zones or segments than described, for example to accommodate special needs for a particular procedure.

The working zone 8 typically is surrounded by a plurality of spring-type coils 12. The coils may be coated with special coatings, such as a hydrophilic or hydrophobic coating 14.

Toward a proximal end of the guidewire 1, the guidewire can include one or more markings or other indicia to indicate to the surgeon how far the guidewire has been advanced. For example, the guidewire could include length measurement marks that tell the surgeon the length of the guidewire that is out of sight in the patient's body.

A typical catheter 16 is shown in FIG. 2. The catheter 16 in its most basic sense is a small hollow tube, typically made of plastic, that can be inserted into human arteries or other vessels in a body's vascular system. This hollow structure facilitates delivering liquids and other materials and devices, such as a guidewire, through its inner passage or lumen.

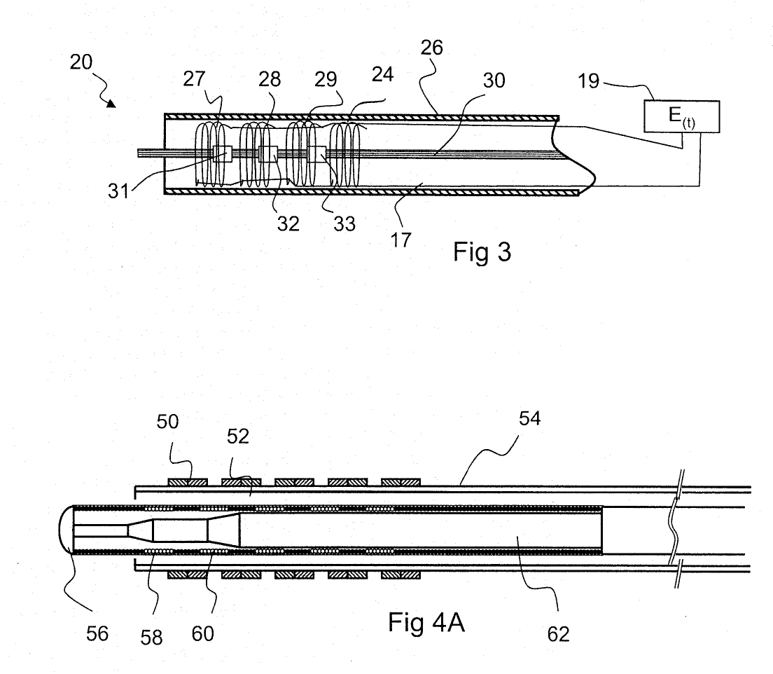

In an exemplary medical device, the guidewire 1 can be vibrated longitudinally, parallel to its longitudinal axis, so that it moves relative to the outer catheter, using a magnetic drive engine. As shown in FIG. 3, a typical magnetic engine can be created by placing fixed magnets on the guidewire in an oscillating magnetic field gradient created by delivering an oscillating electrical current from a generator 19 to magnetic field-generating coils within the catheter via leads 17. In particular, the medical device 20 shown in FIG. 3 includes a catheter 26 with several coils 27, 28, 29, and 24 positioned near its distal end and arranged in a Helmholtz-like configuration. A guidewire 30 extends through the passage in the catheter and has a plurality of magnets, 31, 32, and 33 attached to or incorporated into the guidewire. The location of the coils 27, 28, 29, and 24 and the magnets 31, 32, and 33 are selected to place the array of coils adjacent to the corresponding array of magnets. For example, coils 27 and 28 may surround magnet 31, coils 28 and 29 may surround magnet 32, etc.

Upon excitation of the coils, a magnetic field gradient is generated in between the coils. The fixed magnets 31, 32 and 33 react to this magnetic field gradient to apply an axial or longitudinal force to the guidewire 30. The radial forces generally balance each other so the guidewire will remain centered. In the embodiment shown in FIG. 3, if the current in the coils 27 and 29 travels in a clockwise direction, while the current in coils 28 and 24 travels in a counterclockwise direction, the magnets, which may be positioned such that the north poles of magnets 31 and 33 are at the distal side of the magnets while the north pole of magnet 32 is at a proximal side thereof, then using such an arrangement achieves a multiplication of the magnetic force operating on a single magnet. The multiplication factor is determined by the number of coil and magnet segments. A different number of coil/magnet segments, other than the three segments shown in FIG. 3 may be employed for greater or lesser effect. The magnets in the oscillating magnetic field gradient will vibrate, moving the guidewire forward and backward along its axis.

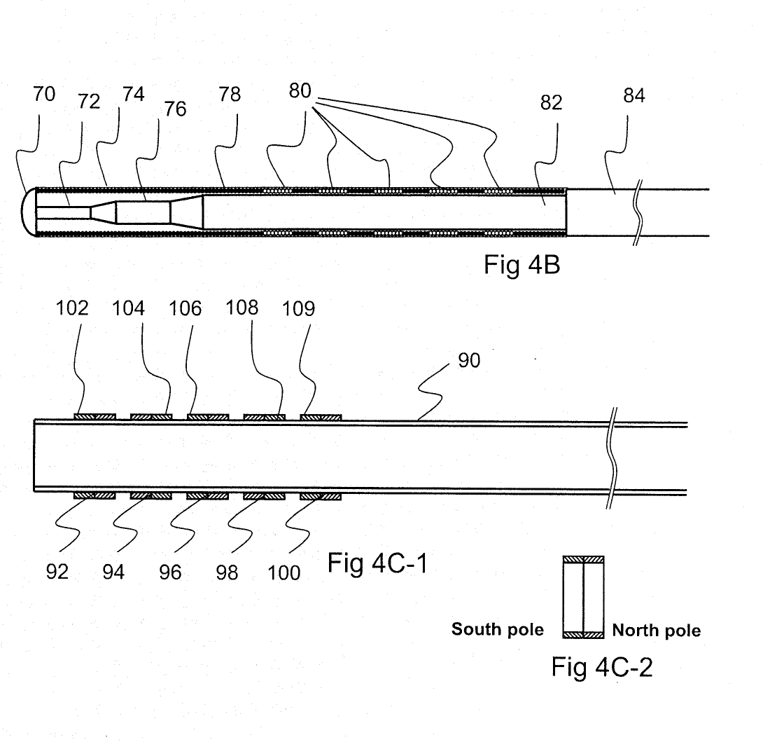

Another way of accomplishing the same effect is shown in FIG. 4A. In this embodiment, tubular magnets 50 or magnetic rings are mounted outside a catheter 54. The guidewire 62 passing through the catheter 54 and magnetic field-generating coils 58 and 60 at the distal end of the guidewire 62 can be energized to generate a magnetic field gradient that reacts with the magnetic fields of the magnets 50 to apply a longitudinal or axial force on the guidewire 62. The guidewire 62 is shown by itself in FIG. 4B. The coils 80 are electrically connected to each other and can transmit electrical current to generate a magnetic flux. As with the previous guidewire, the guidewire 62 includes a distal tip 70, followed by a flexible zone core segment 72, followed by a thicker core segment 76 in the stent zone, and finally a thicker core segment 82, followed by the body of the guidewire 84. The coils covering the different zones are no longer unified and passive as in the earlier embodiment, since the guidewire now includes coil sections 80 for generating a magnetic field. As in the earlier embodiment, the coils 78 can be coated with a bio-compatible material 74 that facilitates the operation. FIG. 4C shows the magnetic catheter and FIG. 4C-2 shows a hollow cylindrical (tubular) magnet with north and south magnetic poles on opposite faces of the cylindrical tube. The magnets typically will be made of rare earth magnetic materials, such as Neodymium Iron Boron (NdFeB) 48. A series of such magnetic rings or cylinders are attached to the catheter as shown in FIG. 4C-1. In this figure, five sets of rings 92, 94, 96, 98, and 100 are attached the outside of the catheter 90. In this embodiment the direction of the magnetic rings includes faces 102, 104, 106, 108, and 109 as the south poles of the magnets, while the opposite face of each magnet is a north pole. Where the magnets overlap one or more magnetic field-generating coils, this overlap defines an active zone of the magnetic drive engine. If the magnets and the coils do not overlap, the engine might not operate. Multiple magnetic drive engines may be provided, connected in series, in parallel, or a combination of series and parallel to provide the desired axial and radial force. Further description of such a magnetic engine can be found in International Application No. PCT/IL2006/000541, which is hereby incorporated herein by reference.

### Configuration 1: Passive Outer Catheter, Coils in Inner Catheter, Magnets on Guidewire

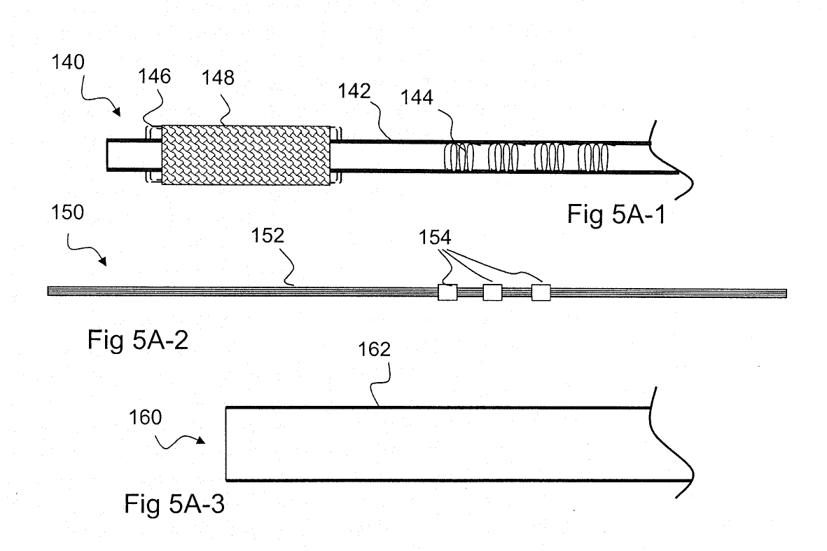

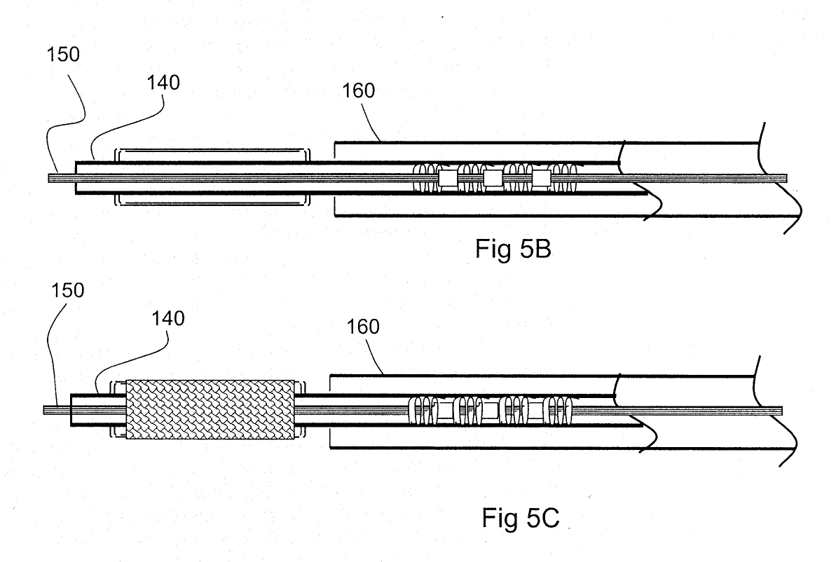

A first embodiment for an exemplary medical device is shown in FIGS. 5A-1-FIG. 5C. FIGS. 5A-1-5A-3 show each of the individual components, which are then combined in FIGS. 5B and 5C. In FIG. 5A-1, an inner catheter 142 is shown carrying a treatment device 140, which may include a balloon 146 and/or a stent 148, for example, both positioned near the distal tip of the catheter 142. The inner catheter 142 further includes one or more magnetic field-generating coils 144, one component of the magnetic drive engine as described above. FIG. 5A-2 shows a guidewire 150 that includes a body 152 on which one or more magnets 154 either are attached to or incorporated into the guidewire. A passive outer or guiding catheter 160 is shown in FIG. 5A-3 and includes a hollow tube 162 with a diameter larger than that of the inner catheter 140. Consequently, when assembled as shown in FIGS. 5B and 5C, the guidewire 150 is housed inside the inner catheter 142, generally parallel to the longitudinal axis of the inner catheter 142, which in turn lies generally parallel to the longitudinal axis of the outer catheter 160. Thus the magnets 154 are positioned in the active zone of the coils 144 that are attached to the inner catheter 140. The outer catheter 160 covers the entire engine so that when the engine is activated and the guidewire 150 oscillates back and forth, the components of the engine are shielded from the walls of the vascular vessel by the outer catheter 160.

The guidewire 150 may be equipped with magnetic beads that are added externally to the guidewire, partially embedded into the guidewire, or fully imbedded into the guidewire. The magnets can be embedded in or otherwise secured to a sterile sleeve that is mounted over a guidewire selected by the surgeon, such as by using a heat-shrinkable or light-shrinkable material, an adhesive, or with a mechanical press-fit, either just before a procedure or as part of the guidewire manufacturing process. Typically the diameter of the guidewire will be about 14 to about 18 mils. The catheter typically has an inner passage with a diameter in the range of about 18 to about 40 mils.

The catheter may have a fixed inner diameter, or it may change diameter along its length. The catheter also can be tapered so that the inner diameter near its distal end is smaller than the inner diameter near a proximal end of the catheter. The coil in the catheter may be external to either catheter, internal to either catheter, or embedded into the walls of either catheter. These coils typically would be made of copper or silver or other electrically conductive material, with a wire diameter typically ranging from about 25 to about 200 microns. The outer or active guiding catheter has a typical diameter of about 1.8 to about 2.1 millimeters. The inner diameter of the catheter in the vicinity of the coils is typically not less than about 1.5 millimeters. Each coil typically has one to four loops, although one embodiment has coils with about twenty-eight turns per layer and two layers, for a total of fifty-six turns. These coils may be coated with an electrical insulating material, a bio-compatible coating, and/or with a thermally conductive coating to improve heat dissipation from the coils in a desired direction.

The relative positions of the magnets and the coils along the guidewire and the catheter can differ from one embodiment to another. Typically, the coils will be spaced approximately 20 to 200 millimeters from the distal tip of the guidewire so that the coils do not change the mechanical characteristics of the leading segments of the guidewire. A wide range is desired to enable the components to overlap without the components of the engine ever extending beyond the distal end of the outer catheter. Thus the distance from the distal tip of the medical device to the magnetic engine can vary from short (about 20 mm) to long (about 200 mm). By varying this distance, the “reach” of the device can be varied. This can be helpful, for example, to keep the engine in the aorta while providing enough “reach” to access a blockage with the tip of the guidewire. The particular coils that are energized can be selected to select how far the guidewire extends from the distal end of the inner catheter as may be desired for a specific vascular vessel or particular blockage or occlusion in the vessel.

### Configuration 2: Passive Outer Catheter, Magnets on Inner Catheter, Coils on Guidewire

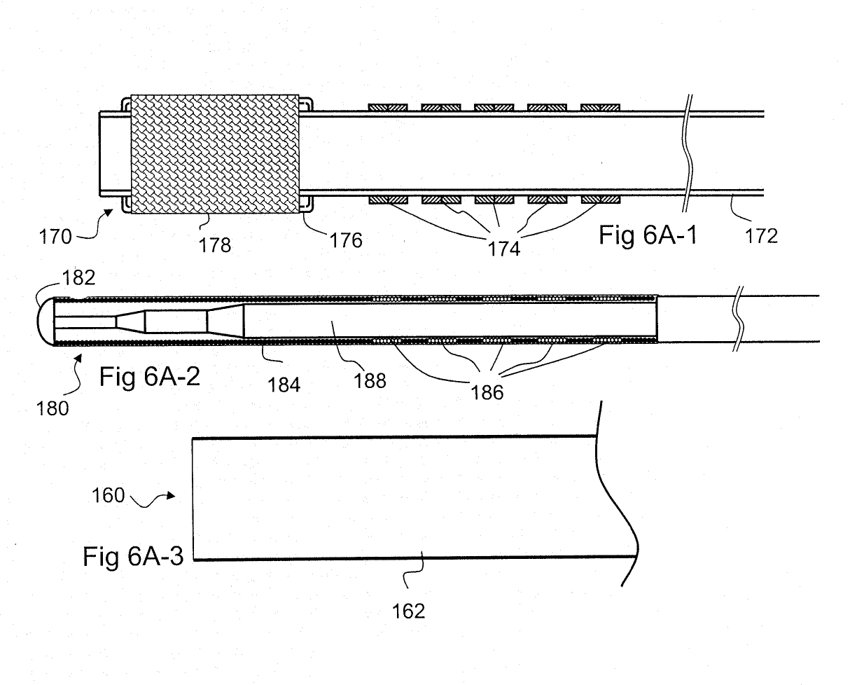

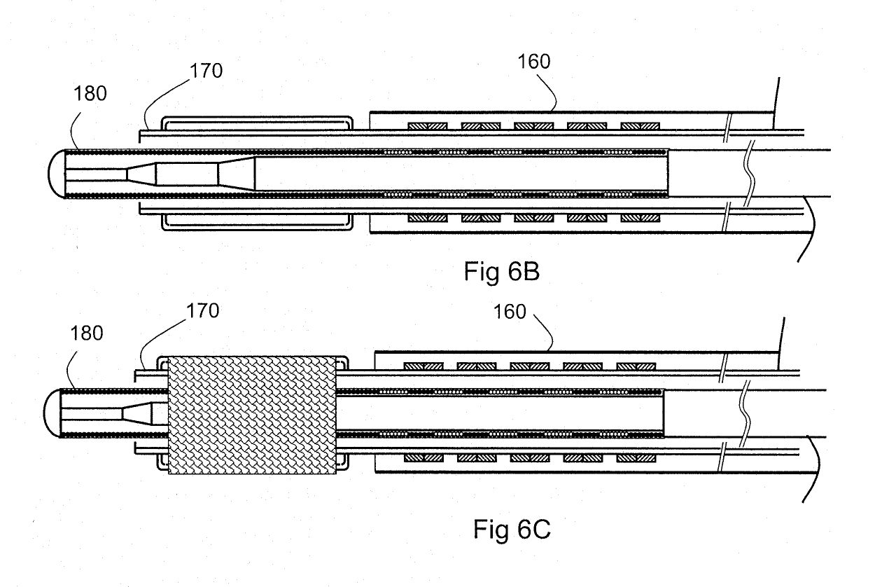

Another exemplary embodiment of the medical device provided by the present invention is shown in FIGS. 6A-1-FIG. 6C. This embodiment is substantially similar to the previous embodiment, except the position of the coils and magnets have been switched and the number of coils and magnets has changed. In this embodiment, an inner catheter 172 has a plurality of magnetic rings 174 secured to its outer surface. The inner catheter 172 also carries a treatment device 170 for delivery to a desired location in a patient's body, including a balloon 176 and a stent 178, for example. (FIG. 6A-1.) The guidewire 180 has a leading or distal tip 182, a core 188, and a series of coils 184 covering the core 188. The coils 184 include a series of spaced electrically-conductive magnetic field-generating coil segments 186 (FIG. 6A-2). Once assembled, the guidewire 180 is telescopically threaded through the inner catheter 172, which is telescopically threaded through a longitudinal passage 162 in an outer catheter 160 (see FIG. 6A-3 and assemblies in FIGS. 6B and 6C). In assembling this device, of course, the guidewire 180 is fed into the vascular system of the patient, and then the inner catheter 172 and the outer catheter 160 are fed into the patient over the guidewire. Once again, the outer catheter 160 completely covers the components of the magnetic engine to protect the walls of the vessel.

### Configuration 3: Coils in Outer Catheter, Passive Inner Catheter, and Magnets on Guidewire

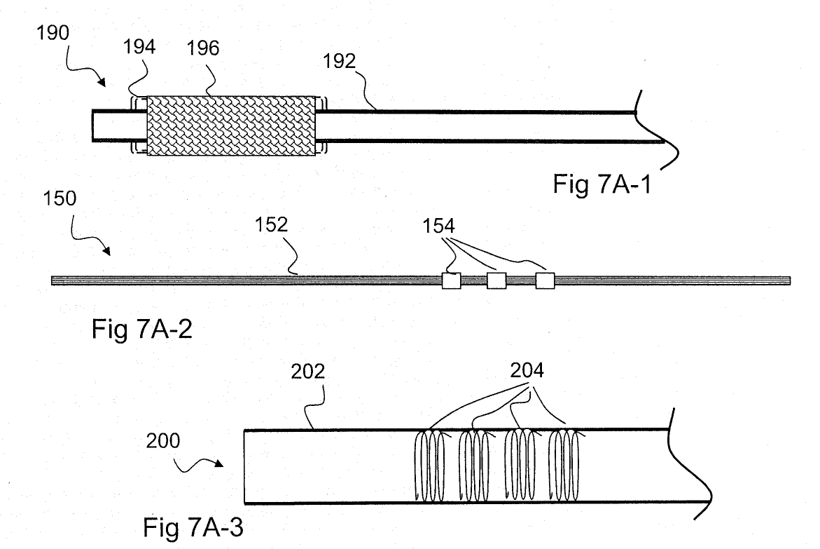

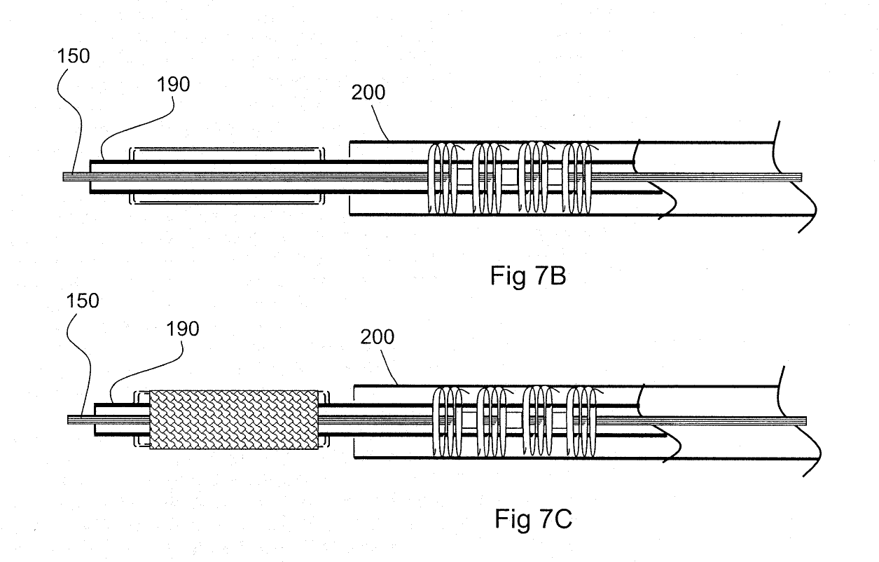

Another alternative embodiment of the medical device provided by the present invention is shown in FIGS. 7A-1-FIG. 7C. FIG. 7A-1 shows a passive inner catheter 192 and a treatment device 190 including a balloon 194 and a stent 196 toward a distal end of the inner catheter 192. In this embodiment, the inner catheter 192 is passive, it does not include any of the components of the magnetic engine. The guidewire 150, shown in FIG. 7A-2 includes a plurality of magnets 154 mounted to or integral with the body of the guidewire 152. The other part of the magnetic engine, coils 204, are shown in FIG. 7A-3 as part of the outer or guiding catheter 200. The coils 204 are mounted inside the outer catheter 200 or are embedded in the walls of the outer catheter 200, but do not extend outside the outer catheter. As shown in FIGS. 7B and 7C, the guidewire 150 extends through the inner catheter 190, and the inner catheter 190 is housed within the outer catheter 200. When the coils 204 are energized, they generate a magnetic field gradient that interacts with the magnets 154 on the guidewire 152. In this case, the internal catheter 190 is passive but separates any rough surfaces of the coils 204 and magnets 154 from each other and from the vessel walls so that the guidewire can move relative to the outer catheter unimpeded and without risking damage to the vessel walls.

Configuration 4: Another Passive Inner Catheter, with Magnets in the Outer Catheter and Coils on the Guidewire.

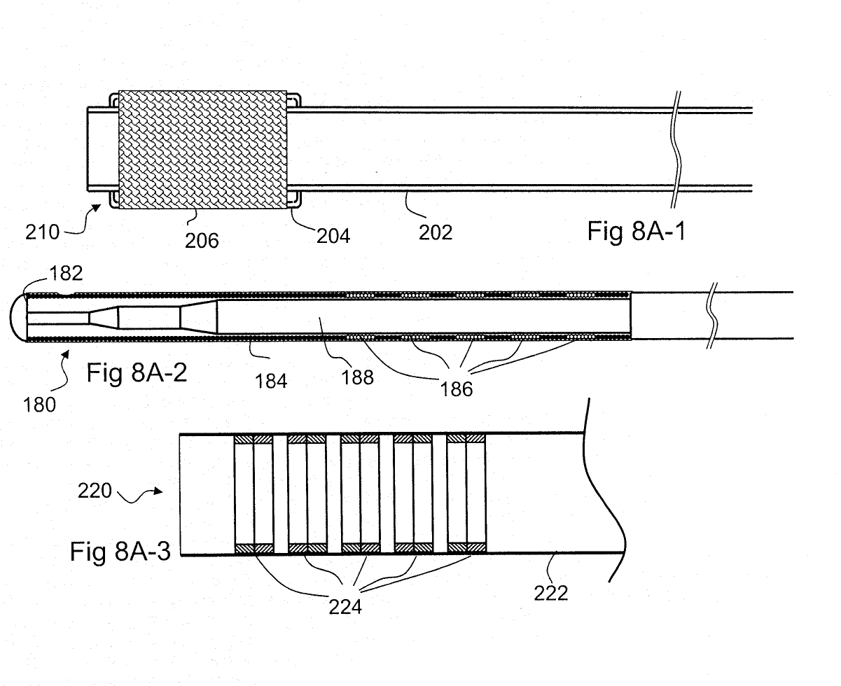

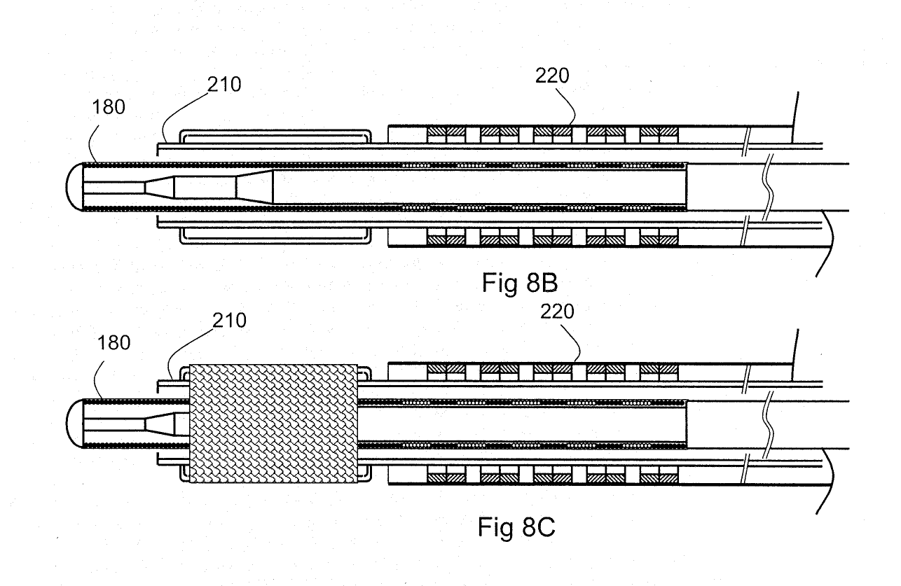

Yet another embodiment provided by the present invention is shown in FIGS. 8A-1-8C. In this embodiment, the inner catheter 202 is again passive and supports a treatment device 210 including a balloon 204 and optionally a stent 206 on the inner catheter 202, for example. The guidewire 180, shown in FIG. 8A-2, includes a core 188, a tip 182, and a spring-like outer sheath 184 that includes multiple integral magnetic field-generating coil segments 186. In this embodiment, the outer or guiding catheter 220 includes ring or tubular magnets 224 mounted inside the outer catheter 222. The assembled device is shown in FIGS. 8B and 8C. As discussed above, the permanent magnets and the coils, the two active components of the magnetic engine, may be applied to any of the guidewire, the inner catheter or the outer catheter in any combination to produce a controllable magnetic flux to drive the oscillatory motion in the guidewire or inner catheter relative to the outer catheter.

### Configuration 5: Passive Guidewire, Magnets on Inner Catheter and Coils in Outer Catheter

As seen in FIGS. 9A-1 through 9C, the magnetic drive engine also can be mounted to the inner and outer catheter for use with a passive guidewire. An advantage of a passive guidewire is that the surgeon can select the guidewire that provides the feel that the surgeon prefers. The surgeon also can select a guidewire having the desired properties for a particular procedure, including the softness/stiffness of the guidewire, whether it is hydrophilic or hydrophobic, etc. To generate the oscillating vibrations in the guidewire, this device also includes a coupling mechanism, described in further detail below.

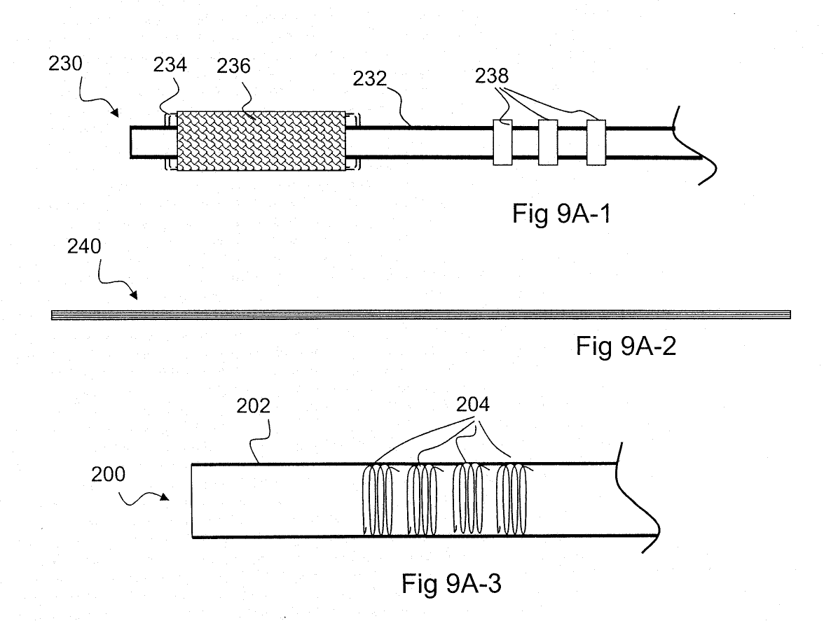

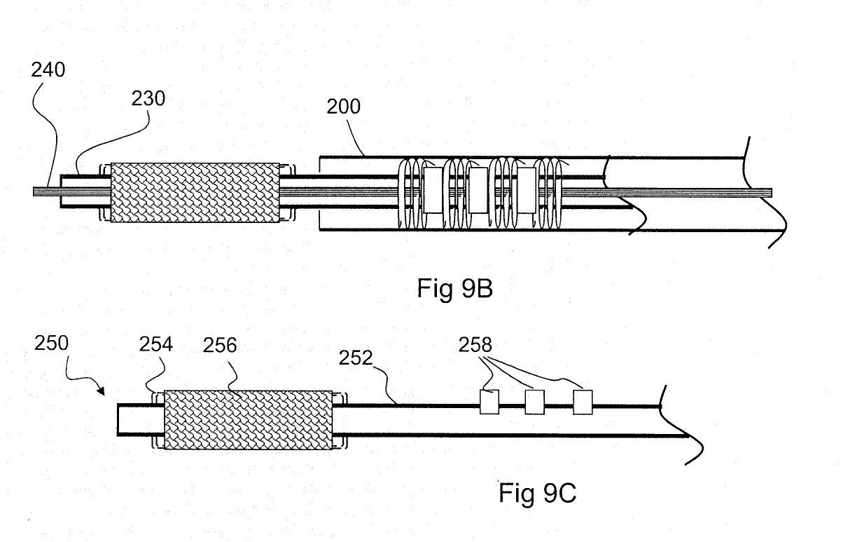

The inner catheter 232 (FIG. 9A-1) optionally can carry a treatment device 230, including a balloon 234 and a stent 236, for example, to a desired location in the patient's body. Magnets 238 are mounted to the outer surface of the inner catheter 232. The guidewire 240 (FIG. 9A-2) is passive, and the surgeon generally can select any guidewire he or she prefers. The outer catheter 200 (FIG. 9A-3) includes a series of magnetic field-generating coils 204 inside the catheter 202. The medical device is assembled in the usual way, with the guidewire 240 threaded through the inner catheter 230, which in turn is threaded through the outer catheter 200 (see FIG. 9B).

The coupling mechanism enables the surgeon to couple and uncouple the guidewire 240 from the active inner catheter 230. When the guidewire is coupled to the inner catheter, the vibratory motion of the inner catheter is transferred to the guidewire, and when the guidewire is uncoupled from the inner catheter, the guidewire will have the sensory feedback, or “feel,” that enables the surgeon to advance the guidewire with minimal danger to the walls of the vessel.

An alternative inner catheter 252 is shown in FIG. 9C. Here the coupling mechanism is provided by off-center magnets 258 mounted to one side of the inner catheter 252. As a result, the force exerted on the magnets in the magnetic field generated by the coils 204 in the outer catheter 200 is not purely co-axial, but includes radial force components as well. The radial force component tends to grip the guidewire within the internal catheter.

As another option (not shown), the inner and outer catheters can be combined into a single catheter having at least two passages (lumens), preferably coaxial passages, that can move relative to one another. The inner passage is equipped with magnets and the outer passage is wrapped with coils, or vice versa.

### Configuration 6: Passive Guidewire, Magnets in Outer Catheter and Coils in Inner Catheter

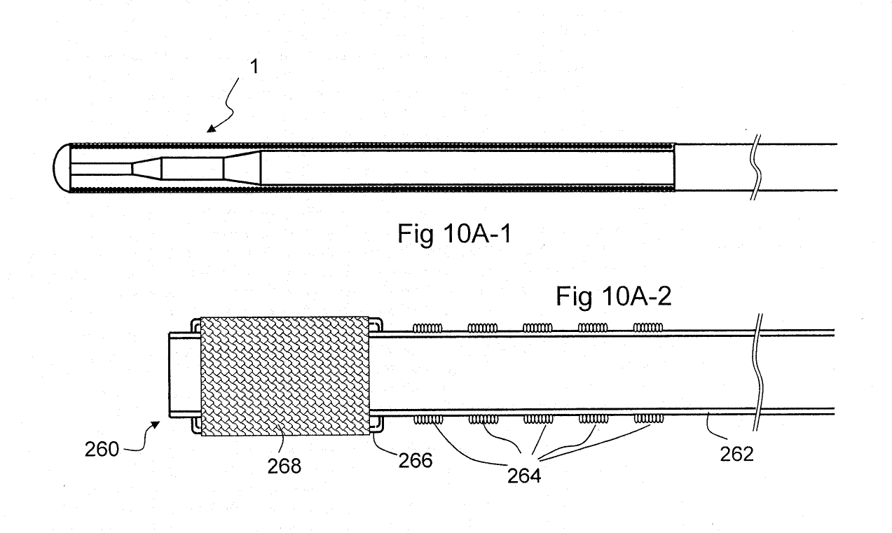

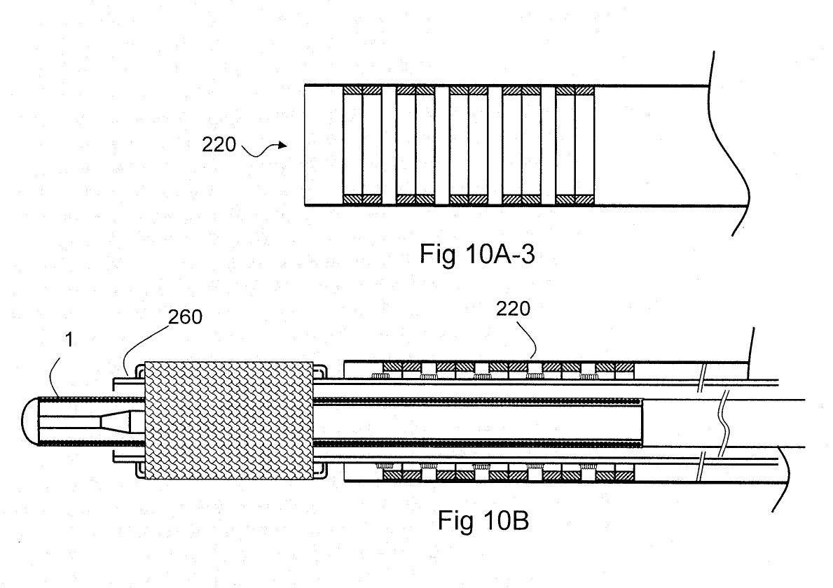

The medical device shown in FIGS. 10A-1 through 10B has a passive guidewire 1, an inner catheter 260 having a plurality of coils 264, an outer catheter 220 with magnets, and a coupling mechanism (not shown).

### Inner Catheter-Guidewire Coupling Mechanism

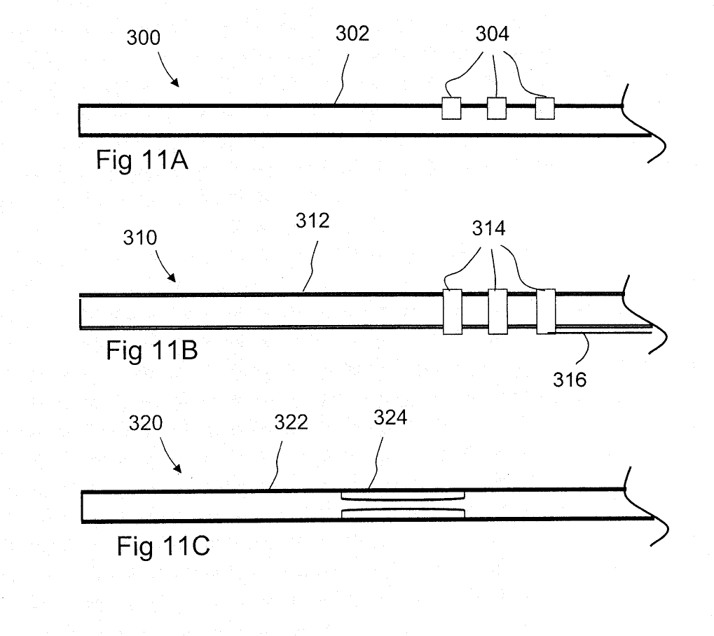

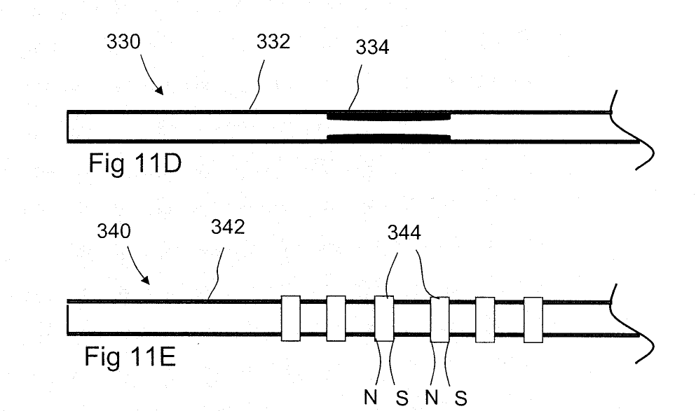

In conjunction with a passive guidewire, various techniques may be employed to couple an active inner catheter to the guidewire to transfer the oscillatory translation or vibration to the guidewire. Some inner catheters with various types of coupling mechanisms are shown in FIGS. 11A-11E.

In FIG. 11A, the inner catheter 300 includes one or more magnets mounted to one side of the catheter to apply both coaxial and radial force components to the catheter and thus the guidewire.

As seen in FIG. 11B, the inner catheter 310 includes a mechanical coupling mechanism that is controlled from a proximal side of the catheter using a mechanical coupling 314 and a control lead 316. Pulling on the lead can engage or disengage the mechanical coupling mechanism that connects the inner catheter to the guidewire. The lead also can function as an electrical lead for coils.

Another coupling mechanism, shown in FIG. 11C, includes a sleeve 324 inside the inner catheter 320 that can be controllably expanded and retracted to reduce or expand the passage through the catheter 320 and grip the guidewire. Preferably the sleeve can be repeatedly expanded and retracted. The sleeve can be activated by electricity, heat, or cold, and returns to its natural expanded or retracted condition when the activating impulse is removed. Alternatively, the sleeve can act as a balloon that is inflated and deflated by a fluid.

The coupling mechanism of FIG. 11D includes an array of tiny rods or “hairs” 334 attached to the passage through the inner catheter 330. These rods are made of a ferromagnetic material and normally are attached to the inner catheter so that they generally lie parallel to and close to the inner walls of the catheter so that they do not interfere with movement of the guidewire. When a magnetic field is applied, however, the rods rotate to stand at an angle closer to perpendicular to the catheter walls, thereby minimizing the diameter of the passage and serving to engage the guidewire and transfer the vibratory motion of the inner catheter to the guidewire. When the magnetic field is removed, the rods return to their original position to release the guidewire. This coupling mechanism has the advantage that creating a magnetic field to generate vibrations simultaneously couples the guidewire to the inner catheter.

In the coupling mechanism of FIG. 11E, the magnets 344 deform the tubular nature of the passage through the guidewire 342 to couple the inner catheter to the guidewire. The deformation can be generated when applying the vibratory magnetic field, so no additional steps need to be taken to couple or uncouple the inner catheter and the guidewire.

One or more of these coupling mechanisms can be used to couple an active inner catheter to a passive guidewire of the surgeon's choosing. The coupling mechanism also can be mounted at different positions on the inner catheter.

### Engine Stop

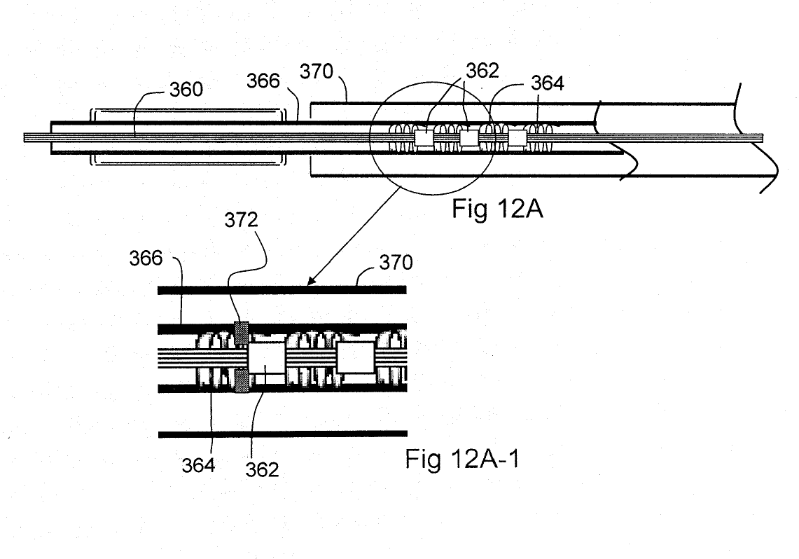

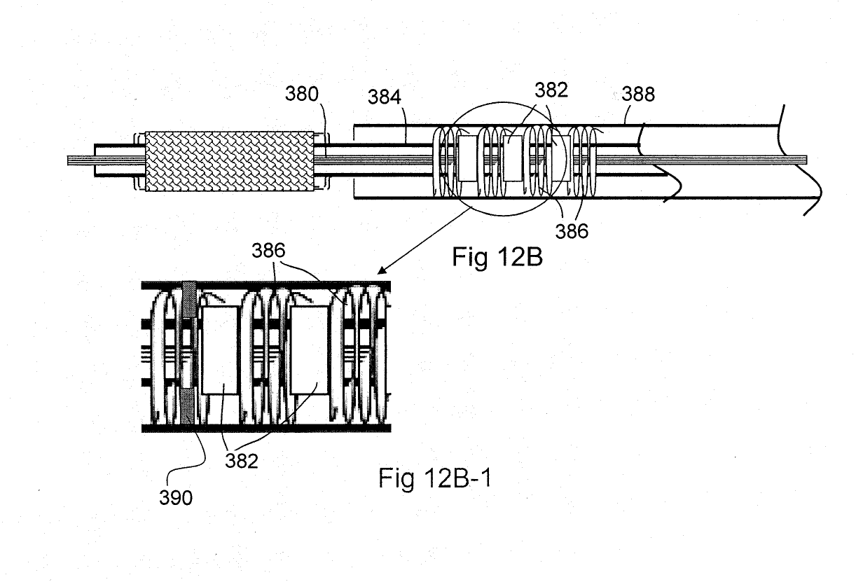

FIGS. 12A, 12A-1, 12B and 12B-1 show two types of stops used to limit the motion of the active inner catheter or guidewire relative to the outer catheter to maintain the active zone in an area of overlapping coils and magnets. If the stops are not retractable, they also can limit the reach of the guidewire beyond the end of the outer catheter.

In FIGS. 12A and 12A-1, the guidewire 360 has a magnets 362 that interact with coils 364 in an inner catheter 366 inside an outer catheter 370. The have a diameter that is larger than the diameter of the guidewire. A stop 372 inside the inner catheter has a central opening through which the guidewire can pass, but is too small for the magnets to pass through, effectively limiting how far the guidewire can advance.

In FIGS. 12B and 12B-1, the guidewire 380 is passive. Magnets 382 are mounted to the inner catheter 384 to interact with coils 386 inside the outer catheter 388. The magnets have an outer diameter that is larger than the outer diameter of the inner catheter. In this arrangement, the stop 390 narrows the passage in the outer catheter enough to allow the inner catheter to pass but not the magnets. The stop thus limits how far the inner catheter can advance.

In both instances, the stops keep the magnets within the magnetic field gradient that can be generated by the coils.

### Piston-Coupled Guidewire

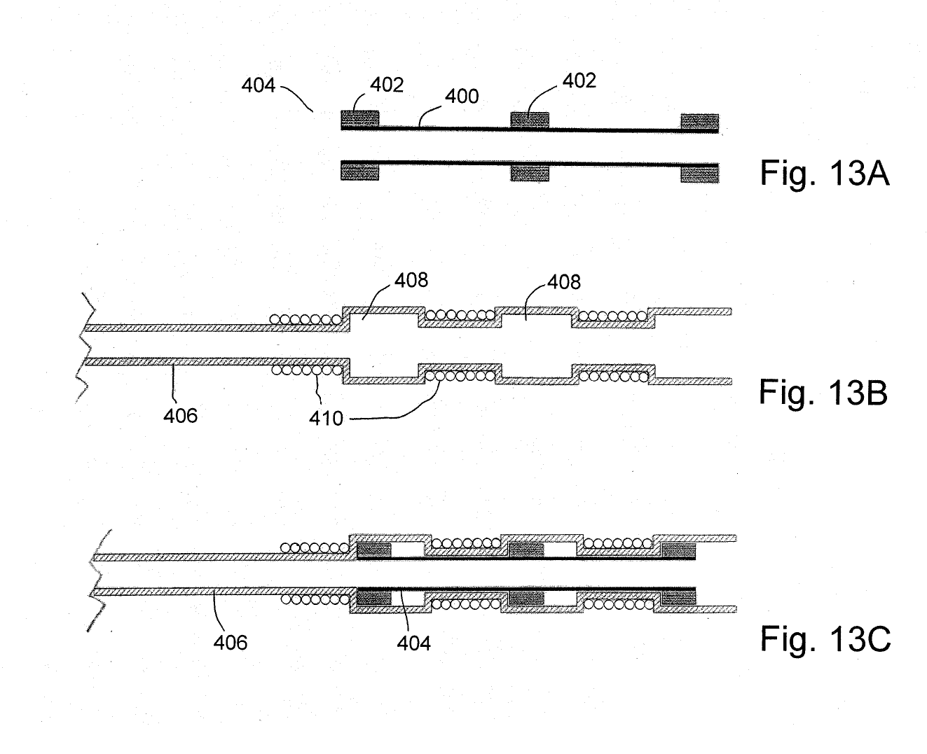

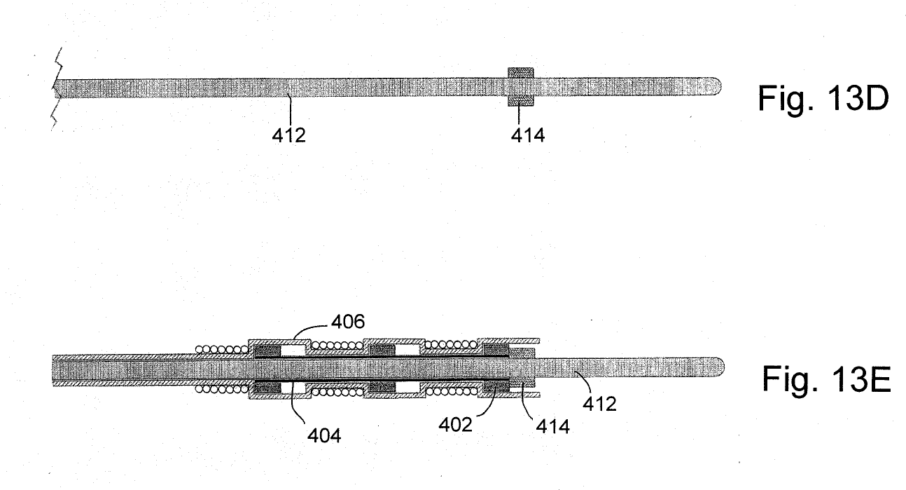

FIGS. 13A-13E illustrate a medical device provided by the present invention with a unique form of stop. In this embodiment an inner catheter 400 with spaced magnets or magnetic rings 402 defines an active piston 404. An outer catheter 406 includes axial slots or annular grooves 408 that receive the magnets therein. These slots in the outer catheter and the magnets on the piston allow the piston to move longitudinally only as far forward and back as the slots allow the magnets, the ends of the slots effectively interfering with further movement and physically preventing the piston from moving out of the active zone. Coils 410 between these slots and separated by the slots can be used to draw the magnets forward and back. Because of the irregular outer surface of the outer catheter, it may be beneficial to insert this outer catheter in a further outer catheter with a smooth outer surface to protect the walls of the vessel. This medical device further includes a guidewire 412 with a magnet 414 coupled to the guidewire. This magnet couples the guidewire to the piston via magnetic attraction to the magnets at one end of the piston. Driving the coils with oscillating electric current generates oscillating axial or longitudinal movement of the piston, which is transferred to the guidewire via this magnetic coupling.

### Vibrating Catheter

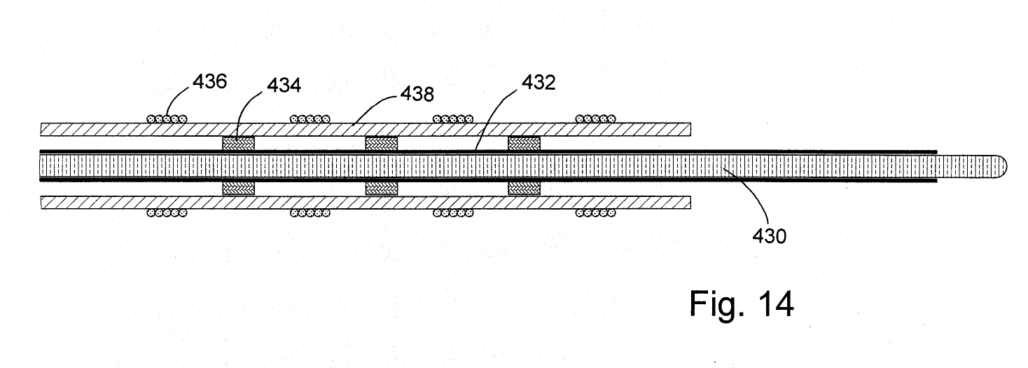

As shown in FIG. 14, a passive guidewire 430 is telescopically inserted into an inner catheter 432 in the form of a thin-walled, flexible hypo-tube with magnets 434 attached to an outer surface. Corresponding coils 436 are mounted inside an outer catheter 438, and of course the positions of the coils and the magnets can be switched, as is apparent from the preceding text. With a thin-walled tube and a small guidewire, the hypo-tube can be about 0.018 inch in diameter. An exemplary tube is made of nitinol. Various patterns of cuts or slits may be made in the tube to further increase its flexibility if desired. The magnets are spaced from the distal end of the tube so that the end of the tube can extend from the outer catheter. Activation of the electromagnetic coils in the outer catheter creates oscillating, axial magnetic forces on the magnets, causing vibration of the hypo-tube. Radial forces also can be applied to the magnets. The vibrating hypo-tube is advanced through a blockage. The surgeon can advance the vibrating hypo-tube, instead of a vibrating guidewire, to the distal side of the blockage. The guidewire can then be advanced through the hypo-tube to the distal side of the blockage. The guidewire holds its position in the blockage while the hypo-tube is withdrawn and exchanged for a treatment device, such as a balloon or stent.

The size of this type of medical device is greatly restricted by the environment in which it operates. It is a particular challenge to provide an effective magnetic engine in such a long and thin device. Accordingly, a catheter-within-a-catheter system such as we have developed would not have been obvious in view of our size constraints.

Although the invention has been shown and described with respect to a certain illustrated embodiment or embodiments, equivalent alterations and modifications will occur to others skilled in the art upon reading and understanding the specification and the annexed drawings. In particular regard to the various functions performed by the above described integers (components, assemblies, devices, compositions, etc.), the terms (including a reference to a “means”) used to describe such integers are intended to correspond, unless otherwise indicated, to any integer which performs the specified function (i.e., that is functionally equivalent), even though not structurally equivalent to the disclosed structure which performs the function in the herein illustrated embodiment or embodiments of the invention.

1. An apparatus, comprising:
an outer catheter having a longitudinal axis;
a movable device that can pass through the catheter and is movable along the longitudinal axis relative to the catheter; and
a magnetic drive engine toward a distal end of the catheter that includes at least two components that move relative to one another, and one of the two components is coupled to the movable device for driving vibratory motion in the movable device along the longitudinal axis.

2. An apparatus as set forth in claim 1, where the movable device includes a guidewire.

3. An apparatus as set forth in claim 1, where the movable device is couplable to the guidewire for movement with the guidewire.

4. An apparatus as set forth in claim 1, where the movable device includes an inner catheter telescopically inserted in the outer catheter, and a guidewire that is telescopically inserted in the inner catheter, the inner catheter including one of the components of the engine and means for selectively coupling the inner catheter to the guidewire.

5. An apparatus as set forth in claim 1, comprising a treatment device selectively coupled to the movable device to deliver the treatment device to a desired location in a patient's body.

6. An apparatus as set forth in claim 1, comprising a stop that limits the relative distal positions of the components of the engine.

7. A device as set forth in claim 1, where the components of the engine have longitudinally-overlapping portions that define an active zone and a stop is positioned to maintain the active zone when the movable device is displaced in a distal direction.

8. A device as set forth in claim 7 that depends from claim 7, where the stop includes an element that is secured to the outer catheter to reduce the diameter of the passage through the outer catheter.

9. A device as set forth in claim 8, where the stop includes a restriction at the distal end of the outer catheter.

10. A device as set forth in claim 7, where the stop includes an element of the guidewire that increases the diameter of the guidewire.

11. A device as set forth in claim 10, where the stop includes an element that is attached to the guidewire to increase its diameter at a location spaced from a distal end of the guidewire by a distance that corresponds to the desired maximum distance that the guidewire can extend beyond the distal end of the outer catheter.

12. A device as set forth in claim 7, comprising an inner catheter telescopically inserted into the outer catheter, where the guidewire is telescopically inserted into the inner catheter and the inner catheter is selectively couplable to the guidewire, and the stop includes a restriction element that is secured to the inner catheter to reduce the diameter of the passage through the inner catheter.

13. A device as set forth in claim 12, where the stop includes an element of the guidewire that increases the diameter of the guidewire beyond the diameter of the passage past the restriction element.

14. A device as set forth in claim 7, comprising an inner catheter telescopically inserted into the outer catheter, where the guidewire is telescopically inserted into the inner catheter and the inner catheter is selectively couplable to the guidewire, and the stop includes a restriction element that is secured to the outer catheter to reduce the diameter of the passage through the outer catheter, and an element of the inner catheter has a diameter beyond the diameter of the passage through the restriction element.

15. A medical device, comprising
an outer catheter having a longitudinal axis;
an inner catheter telescopically inserted in the outer catheter;
a guidewire telescopically inserted in the inner catheter; and
a magnetic drive engine toward a distal end of the outer catheter to vibrate the guidewire along the longitudinal axis.

16. A device as set forth in claim 15, where the engine includes two components that move relative to one another, one component being coupled to the guidewire and the other component being coupled to the inner catheter.

17. A device as set forth in claim 15, where the magnetic drive engine includes a magnet and a coil for selectively generating a magnetic field to move the magnet relative to the coil.

18. A device as set forth in claim 16, where the magnet is connected to the guidewire and the coil is connected to the inner catheter.

19. A device as set forth in claim 18, where the coil is secured inside the inner catheter.

20. A device as set forth in claim 17, where the magnet is connected to the inner catheter and the coil is connected to the guidewire.

21. A device as set forth in claim 20, where the magnet is connected to an outside surface of the inner catheter.

22. A medical device, comprising
an outer catheter having a longitudinal axis;
an inner catheter telescopically inserted in the outer catheter;
a guidewire telescopically inserted in the inner catheter; and
a magnetic drive engine toward a distal end of the outer catheter to vibrate the guidewire along the longitudinal axis;
where the inner catheter is coupled to the guidewire for longitudinal movement with the guidewire relative to the outer catheter.

23. A device as set forth in claim 22, where the engine includes two components that move relative to one another, one component being coupled to the inner catheter and the other component being coupled to the outer catheter.

24. A device as set forth in claim 23, where the magnetic drive engine includes at least one magnet and at least one coil for selectively generating a magnetic field to move the magnet relative to the coil.

25. A device as set forth in claim 23, where the magnet is connected to the inner catheter and the coil is connected to the outer catheter.

26. A device as set forth in claim 25, where the coil is secured inside the outer catheter.

27. A device as set forth in claim 25, where the magnet is connected to an outside surface of the inner catheter.

28. A device as set forth in claim 23, where the magnet is connected to the outer catheter and the coil is connected to the inner catheter.

29. A device as set forth in claim 23, where the inner catheter is permanently secured to the guidewire.
